(19) United States
(12) Patent Application Publication
(10) Pub. No.: US 2010/0144767 A1
(43) Pub. Date: Jun. 10, 2010

(54) Use of sodium channel blockers for the treatment of visceral pain or pain caused by cancer treatment

(75) Inventors: Kim Noel Fisher, Chestermere (CA); Anh Ho Ngoc, Montreal (CA); Edge Renfeng Wang, Castro Valley, CA (US)

Correspondence Address: FISH & RICHARDSON PC, P.O. BOX 1022, MINNEAPOLIS, MN, 55440-1022, US

(73) Assignee: WEX PHARMACEUTICALS, INC., Vancouver, BC (CA)

(21) Appl. No.: 11/990,743

(22) PCT Filed: Aug. 25, 2006
(86) PCT No.: PCT/US2006/033361

§ 371 Date: May 26, 2009

Related U.S. Application Data
(60) Provisional application No. 60/711,140, filed on Aug. 25, 2005.
(60) Provisional application No. 60/760,927, filed on Jan. 23, 2006.

(51) Int. Cl. A61K 31/519; A61P 25/00

(52) U.S. Cl. 514/267

(57) ABSTRACT

The invention provides methods for treating visceral pain and pain associated with therapy. The compounds useful in the methods of the invention are blockers of sodium ion channels, and in particular compounds that bind to the SS1 or SS2 extracellular mouth of the α-subunit thereof. Particularly useful compounds are saxitoxin and its derivatives and analogues and tetrodotoxin and its derivatives and analogues.

### CROSS-REFERENCE TO RELATED APPLICATIONS

This application claims benefit under 35 U.S.C. §119 to U.S. Provisional Application Ser. Nos. 60/711,140, filed Aug. 25, 2005, and Ser. No. 60/760,927, filed Jan. 23, 2006, the entire contents of which are incorporated herein by reference.

### BACKGROUND OF THE INVENTION

1. Field of the Invention

The invention relates to uses of sodium channel blockers to treat visceral pain and pain associated with therapy.

2. Description of Related Art

Pain may be acute or chronic. Perception of pain can also be divided into three areas; acute nociceptive processing, facilitated pain arising from persistent afferent input (as after tissue injury) and neuropathic pain arising from altered processing after nerve injury. Acute pain can be severe, but lasts a relatively short time. It is usually a signal that body tissue is being injured in some way, and the pain generally disappears when the injury heals. Chronic pain may range from mild to severe, and it is present to some degree for long periods of time. Chronic pain often arises without any detectable injury or persists even when an injury has apparently healed.

Sodium channel blockers are known to be useful to treat pain symptoms in some circumstances. Typical sodium channel blockers include tetrodotoxin, saxitoxin and others. Tetrodotoxin and its significance in the study of excitation phenomena has been reviewed by C. Y. Kao, Pharmacological Reviews, Vol. 18, No. 2, 997-1049 (1966).

Adams, et al., U.S. Pat. Nos. 4,022,899 and 4,029,793 pertain to a local anesthetic composition of tetrodotoxin or deoxytetrodotoxin, and another compound, generally a conventional local anesthetic compound or a similar compound having nerve-blocking properties.

Tetrodotoxin can be used as a local anesthetic and is ten thousand times more powerful than commonly used local non-narcotics, as is discussed by C. Y. Kao and F. A. Fuhrman, J. Pharmacol., 140, 31-40 (1963). Tetrodotoxin preparations in combination with other widely used anesthetics have been noted in U.S. Pat. No. 4,022,899 and U.S. Pat. No. 4,029,793. Use of tetrodotoxin as a local anaesthetic and analgesic and its topical administration is described in U.S. Pat. No. 6,599,906 Ku. The systemic use of Tetrodotoxin as an analgesic is described in U.S. Pat. No. 6,407,088 Dong. This document describes the systemic application of tetrodotoxin in combination with suitable pharmaceutical vehicles to alleviate pain.

U.S. Pat. No. 6,030,974 Schwartz, describes a method of producing local anesthesia in a mammal experiencing pain in an epithelial tissue region. The method includes topically administering to the region, in a suitable pharmaceutical vehicle, an effective dose of a long-acting sodium channel blocking compound. The sodium channel blocking compound of U.S. Pat. No. 6,030,974 can be a formulation of tetrodotoxin or saxitoxin at a concentration of between 0.001-10 mM.

Medications and treatments which are suitable to control pain associated with one medical condition may not be suitable to control pain associated with others. Currently opiates are often used to treat moderate to severe pain conditions but these have a range of disadvantages and alternative medications are needed.

### BRIEF DESCRIPTION OF THE DRAWINGS

FIG. 1 shows the results for the antiinflammation test (A) and the Randall-Selitto test (B) in animal model example 3.

FIG. 2 shows the clinical response to TTX treatment in the second cycle in clinical case #1 (3206).

FIG. 3 shows the clinical response to TTX treatment in clinical case #4 (3210).

### DETAILED DESCRIPTION OF THE INVENTION

The compounds useful in the methods of the invention are blockers of sodium ion channels, and in particular compounds that bind to the SS1 or SS2 extracellular mouth of the α subunit thereof. Particularly useful compounds are saxitoxin and its derivatives and analogues and tetrodotoxin and its derivatives and analogues. Their use to treat a range of visceral pain types and pain arising from therapy is disclosed herein.

### DEFINITIONS

“Pain” means all forms of pain, including but not limited to acute pain, chronic pain, centrally and peripherally derived neuropathic and non-neuropathic pain, nociceptive pain, allodynia, causalgia, hyperpathia, hyperalgesia, hyperesthesia, neuritis, and all other conditions and symptoms which would be considered either colloquially or technically to be “pain”. The artisan of ordinary skill in pain management recognizes that pain may arise from many different causes, be expressed by many different physiological mechanisms, and be perceived by patients in many different ways.

Thus, if the present invention is to be applied to the different kinds of pain mentioned above, it may be that different embodiments of the invention must be used. Therefore, when pain of a particular sort is to be addressed, the approach used in the prior art to treat one sort of pain might or might not be effective against the particular kind of pain newly addressed. For example, alternative embodiments the methods described herein may be needed for treating acute pain, chronic pain, neuropathic pain or non-neuropathic pain. The pain may be experienced by a mammal, and by way of example the mammal may be a human.

In a first embodiment there is disclosed a method for the treatment of visceral pain in a mammal. The method may comprise administering to a mammal in need thereof an effective amount of a sodium channel blocker, which may be a sodium channel blocker that binds to the SS1 or SS2 site of the extracellular region of an alpha subunit of a sodium channel.

In alternative embodiments visceral pain may be associated with chronic pancreatitis, may be perineal pain, pelvic pain, scrotal pain, chest pain, pain of the chest wall, or penile pain. In further alternative embodiments the pain may be may be associated with irritable bowel syndrome, gastrointestinal dyspepsia, interstitial cystitis, gall bladder dysfunction, vulvodynia, urethral syndrome, endometriosis, dysmenorrhea, prostatodynia.

In further alternative embodiments the pain may be inflammatory pain, chronic pain or acute pain, or may be caused by therapy which may comprise operative therapy, chemotherapy or radiation therapy.

With respect to treatment of inflammatory pain, the sodium channel blocker does not have any effect upon the degree of inflammation, but instead has an antinociceptive effect, lessening the perception of pain. This has been demonstrated using TTX and the Randall-Selitto test.

In further alternative embodiments the pain may be chronic pain or acute pain. In some embodiments the method may comprise formulating a medicament comprising the sodium channel blocker.

“Sodium channel blockers” or “sodium channel blocking compounds” encompass any chemicals that bind selectively to a sodium channel and thereby deactivate the sodium channel. In particular they include chemicals which bind to the SS1 or SS2 extracellular domains of an alpha subunit of a sodium channel. Sodium channel blocking compounds that bind to the SS1 or SS2 subunit of a sodium channel, particularly tetrodotoxin and saxitoxin, are found to possess similar pharmaceutical activity (U.S. Pat. No. 6,407,088, hereby incorporated by reference).

Tetrodotoxin (“TTX”), also known as Ti Qu Duo Xin, Puffer Fish toxin, maculotoxin, spheroidine, tarichatoxin, tetrodontoxin, fugu poison and TTX (The Merck Index, 10 .sup.th Ed. (1983)), is a biological toxin found in puffer fish (Tetradontiae). The chemical name is octahydro-12-(hydroxymethyl)-2-imino-5,9:7,10a-dimethano-10aH-[1,3]dioxocino[6,5-d]pyrimidine-4,7,10,11,12-pentol with a molecular formula C₁₁H₁₇N₃O₈ and a molecular weight of 319.27. It is a potent non-protein neurotoxin and an indispensable tool drug for the study of neurobiology and physiology. Tetrodotoxin (TTX) is a marine organic toxin which is mainly found in testicles, ovaries, eggs, livers, spleens, eyeballs, and blood of puffer fish as well as in diverse animal species, including goby fish, newt, frogs and the blue ringed octopus and even in marine alga. It is a known substance and production processes are known. Usually TTX is extracted from marine organisms (e.g. JP 270719 Goto and Takashi). However, besides numerous extraction methods, syntheses of TTX have also described and are well known to those skilled in the art. These are exemplified in, e.g. in U.S. Pat. No. 6,552,191, U.S. Pat. No. 6,478,966, U.S. Pat. No. 6,562,968 and US 2002/0086997, all hereby incorporated herein by reference. TTX is well-described in, for example, Tu, Anthony (Ed.) Handbook of Natural Toxins, Vol. 3: Marine Toxins and Venoms, pp. 185-210 (1988), or Cao, Pharmacol. Rev. 18:997-1049 (1966), also hereby incorporated by reference.

Tetrodoxin's “derivatives and analogues” according to this disclosure are defined in part in U.S. Pat. No. 6,030,974 (incorporated herein by reference) as meaning amino perhydroquinazoline compounds having the molecular formula C₁₁H₁₇N₃O₈. “Tetrodoxin derivatives and analogues” according to this disclosure include the compounds described in U.S. Pat. No. 5,846,975 (incorporated herein by reference) as amino hydrogenated quinazolines and derivatives including, but not limited to, the substances described from column 3, line 40 to column 6, line 40 therein. Specifically exemplified “derivatives and analogues of tetrodotoxin” according to this disclosure include but are not limited to anhydro-tetrodotoxin, tetrodaminotoxin, methoxytetrodotoxin, ethoxytetrodotoxin, deoxytetrodotoxin and tetrodonic acid, 6 epi-tetrodotoxin, 11-deoxytetrodotoxin as well as the hemilactal type TTX analogues (e.g. 4-epi-TTX, 6-epi-TTX, 11-deoxy-TTX, 4-epi-11-deoxy-TTX, TTX-8-O-hemisuccinate, chiriquitoxin, 11-nor-TTX-6(S)-ol, 11-nor-TTX-6(R)-ol, 11-nor-TTX-6,6-diol, 11-oxo-TTX and TTX-11-carboxylic acid), the lactone type TTX analogues (e.g. 6-epi-TTX (lactone), 11-deoxy-TTX (lactone), 11-nor-TTX-6(S)-ol (lactone), 11-nor-TTX-6(R)-ol (lactone), 11-nor-TTX-6,6-diol (lactone), 5-deoxy-TTX, 5,11-dideoxy-TTX, 4-epi-5,11-didroxy-TTX, 1-hydroxy-5,11-dideoxy-TTX, 5,6,11-trideoxy-TTX and 4-epi-5,6,11-trideoxy-TTX) and the 4,9-anhydro type TTX analogues (e.g. 4,9-anhydro-TTX, 4,9-anhydro-6-epi-TTX, 4,9-anhydro-11-deoxy-TTX, 4,9-anhydro-TTX-8-O-hemisuccinate, 4,9-anhydro-TTX-11-O-hemisuccinate).

The typical analogs of TTX possess only ⅛ to 1/40 of the toxicity of TTX in mice, based upon bioassay in mice. It has been observed that the analogues produce joint action, and do not interact adversely. Joint action can be either synergistic or additive. Examples of TTX analogs include novel TTX analogs isolated from various organisms, as well as those that are partially or totally chemically synthesized (see e.g., Yotsu, M. et al. Agric. Biol. Chem., 53(3):893-895 (1989)). Such analogs bind to the same site on the alpha subunit of sodium channels as does TTX.

“Derivatives and analogues” of TTX may include compounds having the general formula I

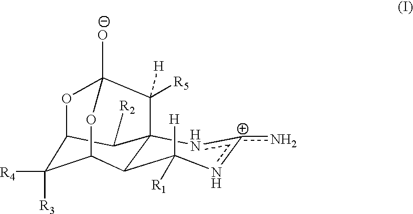

wherein, R² and R⁵ can be selected from the group consisting of H, OH, OAc, respectively;
R¹ can be H, or an alkyl with C₁-C₄, OH, OR, OC(O)R′, NH₂, NHR″, NR″R′″, among them R can be an alkyl with C₁-C₆, R′ can be an alkyl with C₁-C₃, and R″, R′″ can be an alkyl with C₁-C₄, respectively;

### R₃ and R₄ can be ═O, or

when R³ is H, R⁴ can be selected from the group consisting of:
—OR, and R is a branched or straight chain alkyl with C₁-C₇,

### —CH(OH)NHOMe,

### -NAP-gly,

### -NAP-en,

### —CH₂NH₂,

### —CH₂NHCH₃,

### -AAG,

### -NMAG, and

### -ANT;

when R³ is OH or OC(O)R and R is an alkyl with C₁-C₃, R₄ can be selected from the group consisting of:

### —CHO,

—CH₂-gly,

### —CH₂-β-Ala,

### —CH₂-Lys,

### —CH₂-en,

### —CH₂-NAP-Lys

### —CH₂-NAP-en,

—CH(OH)CH(NH₂)COOH; and,
—NH(CH₂)ₙCOOH,
—NH(CH₂)ₙNH₂; and
—NH(CH₂)ₙCH(NH₂)COOH,
wherein:
n=1-6.
en is ethylene;
NAP is 4-triazo-2-nitrobenzoic amide, indicated as formula (a);
AAG is 2-triazo-O-aminobenzoic amide, indicated as formula (b);
NMAG is O-methylaminobenzoic amide, indicated as formula (c);
ANT is O-aminobenzoic amide, indicated as formula (d);

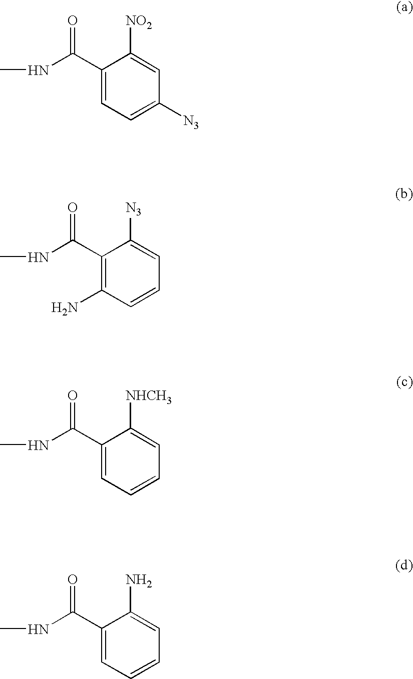

Among compounds of formula (I), in alternative embodiments three kinds of compounds with the general formula II, III, IV may be selected.

In alternative embodiments the amino hydrogenated quinazoline compounds and derivatives thereof may be compounds having following general formula II,

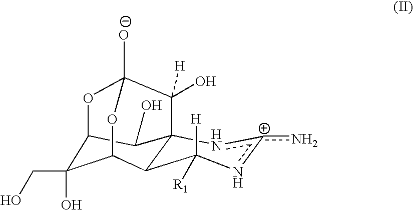

wherein: R¹ can be selected from the group consisting of OH, an alkyl or an oxyalkyl with C₁-C₄, NH₂, NHR″, NR″R′″, among them R″ and R′″ can be an alkyl with C₁-C₄.

Among compounds of formula (II), selected compounds may be:

### Tetrodotoxin R₁═OH (1);

deoxytetrodotoxin R₁═H (2);
The amino hydrogenated quinazoline compounds and derivatives thereof may be compounds having following general formula III

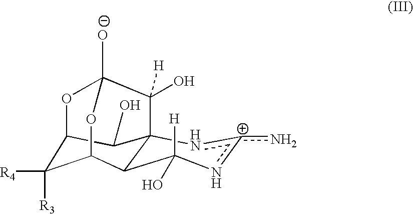

wherein:

### R³, R⁴ are ═O, or

when R₃ is H, R₄ is selected from the group consisting of:

### CH₂OH,

### CH(OH)NHOMe,

### -NAP-gly,

### -NAP-en,

### —CH₂NH₂,

### —CH₂NHCH₃,

### -AAG,

### -NMAG, and

### -ANT.

Among compounds of formula (III), selected compounds may be:

### AAG-degradation Tetrodotoxin R⁴=AAG (3);

### NMAG-degradation Tetrodotoxin R⁴=NMAG (4);

ANT-degradation Tetrodotoxin R⁴=ANT (5); and,
degradation Tetrodotoxin R³, R⁴ is ═O (6).
In alternative embodiments the amino hydrogenated quinazoline and their derivatives may be compounds having following general formula IV,

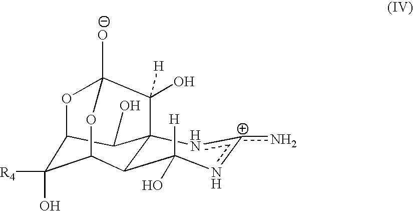

wherein, R⁴ can be selected from the group consisting of:

### —CHO,

### —CH₂-Gly,

### —CH₂-β-Ala,

### —CH₂-Lys,

### —CH₂-en,

### —CH₂-NAP-Lys

### —CH₂-NAP-en,

### —CH(OH)CH(NH₂)COOH,

### —NH(CH₂)₄CH(NH₂)COOH,

### —NHCH₂COOH,

### —NHCH₂CH₂COOH, and

### —NHCH₂CH₂NH₂.

Among compounds of formula (IV), in alternative embodiments, the selected compounds may be:

oxytetrodotoxin R⁴═CHO (7);
chiriquitoxin R⁴═CH(OH)CH(NH₂)COOH (8);
and the compounds with the substituted groups of R⁴:

### —NH(CH₂)₄CH(NH₂)COOH (9);

### —NHCH₂COOH (10);

—NHCH₂CH₂COOH (11); and,

### —NHCH₂CH₂NH_{2 (}12).

Saxitoxin (STX) and its pharmacologically acceptable salts are species of 2,6-diamino-4-((aminocarbonyl)oxy)methyl-3a,4,8,9-tetrahydro-1H,10H-pyrrolo(1,2-c)purine-10,10-diol (3aS-(3a-a-a-4-a,10aR*)). The molecular formula of saxitoxin is C₁₀H₁₇N₇O₄, it has a molecular weight of 299.3 and a general structure of:

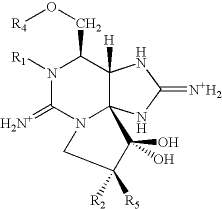

This, and its derivatives and its analogues may be used in accordance with the disclosure. Saxitoxin is readily soluble in water and can be dispersed in aerosols. It is toxic by ingestion and by inhalation, with inhalation leading to rapid respiratory collapse and death. Chemically, saxitoxin is stable, although it can be inactivated by treatment with strong alkali. It is naturally-occurring, produced by bacteria that grow in other organisms, including the dinoflagellates Gonyaulax catenella and G. tamarensis; which are consumed by the Alaskan butter clam Saxidomus giganteus and the California sea mussel, Mytilus californianeus. The toxin can be isolated from S. giganteus or M. californianeus. The first synthesis of STX was completed by Kishi and co-workers at Harvard in 1977 (J. Am. Chem. Soc. 1977, 99, 2818). A second synthesis was carried out by Jacobi and his collaborators whilst at Wesleyan University, Connecticut (J. Am. Chem. Soc. 1984, 106, 5594). A range of alternative methods for the synthesis and purification of saxitoxin will be apparent to those skilled in the art. Analogues and derivatives of saxitoxin include but are not limited to neosaxitoxin and anhydrosaxitoxin, any other biologically active variants of the above saxitoxin structure, and pharmaceutically acceptable salts thereof.

Compounds that are “administered together with TTX” or “in combination with TTX” may be administered as part of the same composition, or may be administered separately, at the same or at separate times, in the same therapeutic regimen.

“Derivatives and analogues” as used in this application has its usual meaning and includes synthetic and biologically derived derivatives and analogues of the compound in question.

The term “neutral form” refers herein to a non-ionic form or to a neutrally charged form (at its isoelectric point) containing an equal amount of positive and negative charges such as for example a zwitterionic species.

The term “salt” according to this disclosure is to be understood as meaning any form of the active compound according to the disclosure in which this compound assumes an ionic form or is charged and—if applicable—is also coupled with a counter-ion (a cation or anion). By this are also to be understood complexes of the active compound with other molecules and ions that are formed via ionic interactions. Preferred examples of salts include acetate, mono-trifluoracetate, acetate ester salt, citrate, formate, picrate, hydrobromide, monohydrobromide, monohydrochloride or hydrochloride salts.

The term “physiologically acceptable salt” in the context of this disclosure is understood as meaning a “salt” (as defined above) of at least one of the compounds according to the disclosure that is physiologically tolerated—especially if used in humans and/or mammals.

The term “solvate” according to this invention is to be understood as meaning any form of the active compound according to the invention in which the compound is attached to another molecule via non-covalent binding (most likely a polar solvent). Particular solvates of the invention include hydrates and alcoholates such as for examples methanolates.

“Synthesis” or “synthesized” has its usual meaning and includes the formation of a compounds through one or more chemical reactions involving simpler components, which simpler components may include biologically derived precursors, or analogues of the compound.

In this application “about” means “approximately,” and illustratively, the use of the term “about” indicates that dosages slightly outside the cited ranges may also be effective and safe, and such dosages are also encompassed by the scope of the present claims.

“Mouse bioassay” refers to the method of assaying the toxicity of a given solution or compound. In the methods used herein the toxicity of raw extracted solution from the extraction chamber or from some other stage in the embodiments was measured in a standard mouse bioassay wherein 0.4 mL of solution desired to be assayed was injected intraperitoneally into laboratory mice with bodyweight of 20 grams. Time to death was measured and material was considered extremely toxic if death occurred in less than 50 seconds, highly toxic if between 50 and 70 seconds, mildly toxic if between 70 and 90 seconds. If death took more than 90 seconds then the toxin content of the liquid was considered not sufficient for further processing. It will be appreciated that a range of alternative assays, using a range of animals or other methods (such as TLC, chromatography, rat bioassays, antibody assays, radioassays and the like) may be useable instead of the mouse bioassay. Suitable methods and choices of protocol will be readily apparent to those skilled in the art.

In this application the term “effective amount” means, consistent with considerations known in the art, the amount of sodium channel blocking agent or other agent effective to elicit a clinically relevant pharmacologic effect or therapeutic effect. In the present invention, this is a reduction in the perception of pain.

It will be appreciated that for the purposes set out herein, tetrodotoxin, saxitoxin, and their derivatives or analogues or metabolite, can be optionally in the form of their racemate, pure stereoisomers, especially enantiomers or diastereomers or in the form of mixtures of stereoisomers, especially enantiomers or diastereomers, in any suitable ratio; in neutral form, in the form of an acid or base or in form of a salt, especially a physiologically acceptable salt, or in form of a solvate, especially a hydrate.

In the context of the embodiments set out herein any amount defined refers to each compound individually not to any combination and refers to the amount of compound present when the compound has a purity of ≧7%. For example, this would mean that a formulation containing 0.5 mg tetrodotoxin of 99% purity and 0.8% anhydro-tetrodotoxin will be classified and defined according to this invention as containing just 0.5 mg tetrodotoxin as active ingredient.

According to the various embodiments, said sodium channel blockers or the pharmaceutical compositions comprising them, may be administered, in unit dosage form, intestinally, enterally, parenterally or topically, orally, subcutaneously, intranasally, by inhalation, by oral absorption, intravenously, intramuscularly, percutaneously, intraperitoneally, rectally, intravaginally, transdermally, sublingually, buccally, orally transmucosally. Administrative dosage forms may include the following: tablets, capsules, dragees, lozenges, patches, pastilles, gels, pastes, drops, aerosols, pills, powders, liquors, suspensions, emulsions, granules, ointments, creams, suppositories, freeze-dried injections, injectable compositions, in food supplements, nutritional and food bars, syrups, drinks, liquids, cordials etc, which could be regular preparation, delayed-released preparation, controlled-released preparation and various micro-granule delivery system. In the case of tablets, various carriers known in the art may be used, e.g. dilutents and resorbents such as starch, dextrin, calcium sulfate, kaolin, microcrystalline cellulose, aluminium silicate, etc; wetting agent and adhesives such as water, glycerin, polyethylene glycol, ethanol, propanol, starch mucilage, dextrin, syrup, honey, glucose solution, acacia, gelatin, carboxymethylcellulose sodium, shellac, methylcellulose, potassium phosphate, polyvinylpyrrolidone, etc; disintegrating agents, such as dried starch, alginate, agar powder, laminaran, sodium bicarbonate and citric acid, calcium carbonate, polyoxyethylene sorbitol aliphatic ester, lauryl sodium sulfate, methylcellulose, ethylcellulose, lactose, sucrose, maltose, mannitol, fructose, various disaccharides and polysaccharides etc; disintegration inhibiting agents, such as sucrose, tristearin, cacao butter, hydrogenated oil, etc; absorption accelerator, such as quaternary ammonium salt, lauryl sodium sulfate, etc; lubricants, such as talc, silica, corn starch, stearate, boric acid, fluid wax, polyethylene, etc. The tablet may be further formulated into a coated tablet, e.g. sugar-coated tablet, film-coated tablet, enteric-coated tablet, or double-layer tablet and multi-layer tablet. In the case of a pill, various carriers known in the art may be used, e.g. dilutents and resorbents, such as glucose, lactose, starch, cacao butter, hydrogenated vegetable oil, polyvinylpyrrolidone, kaolin, talc, etc; adhesives, such as acacia, bassora gum, gelatin, ethanol, honey, liquid sugar, rice paste or flour paste, etc; disintegrating agent, such as agar powder, dried starch, alginate, lauryl sodium sulfate, methylcellulose, ethylcellulose. In case of a suppository, various carriers known in the art may be used, e.g. polyethylene, lecithin, cacao butter, higher alcohols, esters of higher alcohols, gelatin, semi-synthetic glyceride, etc. In the case of a capsule, it may be prepared by mixing said sodium channel blockers as active ingredient with the above mentioned carriers, followed by placing the mixture into a hard gelatin capsule or soft capsule. Also, said sodium channel blockers may be applied in the following dosage forms: microcapsules, suspension in an aqueous phase, hard capsule, or injection. In the case of injection, such as liquor, emulsion, freeze-dried injection, and suspension, all the dilutents common in the art may be used, e.g. water, ethanol, polyethylene glycol, propylene glycol, oxyethylated isostearyl alcohol, polyoxidated isostearyl alcohol, polyoxyethylene sorbitol aliphatic ester, etc. In addition, in order to obtain isotonic injection, a suitable amount of sodium chloride, glucose or glycerin may be added into the preparation, as well as regular cosolvent, buffer, pH adjusting agent, etc. In addition, coloring agents, antiseptics, perfumes, correctives, food sweetening agents or other materials may be added to the pharmaceutical preparation if necessary.

In alternative embodiments the sodium channel blocker may be selected from the group consisting of: tetrodotoxin, saxitoxin, and derivatives or analogues of tetrodotoxin and saxitoxin; may be tetrodotoxin or an analogue or derivative thereof; may be selected from the group consisting of tetrodotoxin, anhydro-tetrodotoxin, tetrodaminotoxin, methoxytetrodotoxin, ethoxytetrodotoxin, deoxytetrodotoxin, epi-tetrodotoxin and tetrodonic acid; or may be tetrodotoxin.

In alternative embodiments the sodium channel blocker may be isolated from a fish, which may be a puffer fish; or may be produced by synthesis or fermentation.

In further alternative embodiments the sodium channel blocker may be administered orally; may be administered sublingually, buccally or transmucosally; may be administered by injection.

In further alternative embodiments the sodium channel blocker may be administered in an amount of between about 5 μg and about 300 μg per unit dose; or between about 5 μg and about 50 μg: or may be administered over a period of between about one and about five days.

The embodiments disclosed may be provided in kit form. Many varieties of kit will be readily envisaged by those skilled in the art, and in particular embodiments comprising kits, components of the disclosed embodiments may be provided in combined or separate form and may be provided along with means for administration such as needles, patches, tablets and other dosage forms. A kit may include instructions on how to use the compositions provided therein and the dosages to be applied.

In particular embodiments the sodium channel blocker may be a voltage-gated sodium channel blocker and may bind to a SS1 or SS2 α subunit of a sodium channel. The maximum daily dose of sodium channel blocker may be up to about 10 μg, up to about 50 μg, up to about 100 μg, up to about 144 μg, up to about 150 μg, up to about 300 μg, up to about 500 μg, up to about 750 μg, up to about 1000 μg, up to about 1250 μg, up to about 1500 μg, up to about 1750 μg, up to about 2000 μg or more. In particular embodiments the sodium channel blocker may be administered in an amount ranging between 5 and 4000 μg/day, or in ranges between 10 and 2000 μg/day, 10 and 1000 μg a day, 10 and 750 μg a day, 10 and 500 μg a day, 10 and 400 μg a day, 10 and 300 μg a day, 10 and 200 μg a day, or 10 and 100 μg/day. In particular embodiments the daily applied dose may be from about 10 to about 160 μg, about 10 to about 140 μg, about 10 to about 120 μg, about 10 to about 100 μg, about 10 to about 90 μg, about 10 to about 80 μg, about 10 to about 70 μg, about 10 to about 60 μg, about 10 to about 50 μg, about 10 to about 40 μg, about 10 to about 30 μg, or 1 to 20 μg.

In alternative embodiments the daily dosage of the sodium channel blocker may be about 0.1 to about 40 μg per kilogram of body weight, about 0.1 to about 20 μg per kilogram of body weight, about 0.1 to about 10 μg per kilogram of body weight, about 0.2 to about 10 μg per kilogram of body weight, about 0.2 to about 5 μg per kilogram of body weight, about 0.5 to about 5 μg per kilogram of body weight, or about 0.5 to about 1 μg per kilogram of body weight.

In certain embodiments an individual dose may be within a range of about 5 μg to about 2000 μg and may be about 5 to about 10 μg, about 10 to about 15 μg, about 15 to about 20 μg, about 20 to about 25 μg, about 25 to about 30 μg, about 30 to about 40 μg, about 40 μg to about 50 μg, about 50 μg to about 75 μg, about 75 to about 100 μg, about 100 to about 150 μg, about 150 to about 200 μg, about 200 to about 250 μg, about 250 to about 500 μg, about 500 to about 1000 μg, about 1000 to about 1500 μg or about 1500 to about 2000 μg or more than 2000 μg.

The sodium channel blocker may be administered in a schedule of one, two, three, four, five, six, seven, eight, nine, ten, eleven, twelve, thirteen, fourteen, fifteen, sixteen, seventeen, eighteen, nineteen, twenty or more doses per day, alone or in combination with other medications, over a range of time periods including but not limited to periods of one, two, three, four, five, six, seven, eight, nine, ten, eleven, twelve, thirteen, fourteen, sixteen, eighteen, twenty, twenty four, thirty, or more days; or over a period of one, two, three, four, five, six, seven, eight, nine, ten, eleven, twelve, thirteen, fourteen, sixteen, eighteen, twenty, twenty four, thirty, thirty six, forty eight, sixty, seventy two, eighty four or more months.

In some embodiments the effectiveness of a course of treatment of one, two, three, four, five or more doses or one, two or three days may last for up to about five, ten, fifteen, twenty, twenty five or thirty days. In some embodiments dosing is only performed once every day or once every two, three, four, five, six, seven, eight, nine, ten, eleven, twelve, thirteen, fourteen, sixteen, eighteen, twenty, twenty four, thirty or more days.

According to the present invention, the dosage of said sodium channel blockers depends on a variety of factors, including the nature and severity of the diseases, the sex, age, weight and individual reaction of the subject, the particular compound employed, the route and frequency of administration, and any other relevant variables. Said sodium channel blockers or the pharmaceutical compositions comprising them may be administered in single or divided dosage form, e.g. one to four doses per day.

A preferred regimen is from 0.2 to 0.8, more preferably 0.2 to 0.4 μg/kg body weight administered once or twice per day orally or by intramuscular injection over a course of one to three days. This regimen may be repeated once per month or once every other month.

Generally the specific and practical ways of preparing the administerable pharmaceutical formulations suitable for use in the embodiments disclosed herein (as well as that of all other formulations mentioned in this disclosure) are well known in the art. Accordingly it is i.a. referred to “Remington: The Science and Practice of Pharmacy”, 21st ed., A. R. Gennaro, et al. Eds., c. 2005 by Lippincott Williams & Wilkins, hereby incorporated in its entirety and for all purposes by reference.

Methods and compositions useful for formulating dosage forms and compositions for use in the embodiments described herein are presented in related filings including: WO 2005/123088 SOLID ORALLY INGESTIBLE FORMULATIONS OF TETRODOTOXIN; U.S. Pat. No. 6,407,088 METHOD OF ANALGESIA; U.S. Pat. No. 6,599,906 A METHOD OF LOCAL ANALGESIA AND ANALGESIA; U.S. Pat. No. 6,559,154 A COMPOSITION OF A SODIUM CHANNEL BLOCKING COMPOUND; WO 2005/004874 A STABLE PHARMACEUTICAL COMPOSITION OF FREEZE-DRIED TETRODOTOXIN POWDER.

In the formulation Examples described below, certain materials are referred to by trade names. In this regard:

POVIDONE K-30 is manufactured by GAF and is a polyvinylpyrrolidone (PVP) of a mean molecular weight of 30,000.

OPADRY II is distributed by Colorcon and is a mixture of polymers, plasticizers and color pigments.

NATROSOL 250 HHX is a hydroxyethylcellulose product of Hercules, Inc., Wilmington, Del. 250 HHX is a grade that is used in long acting tablet formulations.

CAB-O-SIL is an amorphous fumed silica produced by Cabot Corp. Cabosil is an extremely fine particle size silica (silicon-dioxide/SiO2) aerogel. It is pure white and free-flowing. Each volume contains about 94% dead air space, with a density of only 2.3 lb/cu ft. On the other hand, water (density 62.4 lb/cu ft) weighs about 27 times more. M5 is a pharmaceutical grade that is a micronized powder.

SURELEASE is a product of Colorcon, West Point, Pa. and is an aqueous ethylcellulose dispersion.

SURETERIC is a product of Colorcon and is an alternative to acrylic polymer systems for enteric coating of solid oral dosage. SURETERIC is a specially blended combination of PVAP (polyvinyl acetate phthalate), plasticizers, and other ingredients in a completely optimized dry powder formulation.

ACRYL-EZE is a product of Colorcon and is an aqueous acrylic enteric coating.

Simulated intestinal fluid is described in the U.S. Pharmacoepia and is made by dissolving 6.8 g of monobasic potassium phosphate in 250 mL of water. Then 77 mL of 0.2 N potassium hydroxide is added with 500 mL of water. 10.0 g of pancreatin is added and the solution is adjusted to pH 6.8+0.1 with 0.2 N potassium hydroxide or 0.2 N hydrochloric acid. The volume of the solution is then made to 1 L with water.

Simulated gastric fluid is described in the U.S. Pharmacoepia and is made by dissolving 2.0 g of sodium chloride and 3.2 g of purified pepsin from porcine stomach mucosa and having an activity of 800 to 2500 units per mg in 7.0 mL of hydrochloric acid and sufficient water to make 1 L. The solution has a pH of about 1.2.

Examples of typically suitable routes of administration, dosage ranges and administration schedules for use of tetrodotoxin are shown in Table 1.

TABLE 1
Administration of Tetrodotoxin

| Route of Administration | Dose (μg/50 kg subject) | Schedule |
|---|---|---|
| Intramuscular injection | 5-50 | 4~2/day |
| Intravenous injection | 5-30 | 3~2/day |
| Subcutaneous injection | 5-50 | 4~2/day |
| Sublingual | 5-30 | 3~2/day |
| Patch through skin | 5-60 | 4~2/day |
| Oral ingestion | 5-30 | 3~2/day |
| Implantable Osmotic pump | 30-60 | 1 |
| Collagen implants | 30-60 | 1 |
| Aerosol | 5-50 | 4~2/day |
| Suppository | 5-30 | 3~2/day |

Typically, the active ingredient tetrodotoxin or saxitoxin may be formulated into purified water or an acetic acid-sodium acetate buffer as a vehicle. However, the formulation can contain other components, including, but not restricted to, buffering means to maintain or adjust pH, such as acetate buffers, citrate buffers, phosphate buffers and borate buffers; viscosity increasing agents such as polyvinyl alcohol, celluloses, such as hydroxypropyl methyl cellulose and carbomer; preservatives, such as benzalkonium chloride, chlorobutanol, phenylmercuric acetate and phenyl mercuric nitrate; tonicity adjusters, such as sodium chloride, mannitol and glycerine; and penetration enhancers, such as glycols, oleic acid, alkyl amines and the like. The addition of a vasoconstrictor to the formulation is also possible. Combination formulations including the long-acting sodium channel blocking compound and an antibiotic, a steroidal or a non-steroidal anti-inflammatory drug and/or a vasoconstrictor are also possible.

Formulation for each administration route in Table 1 is generally considered known in the art. See, e.g., “Remington: The Science and Practice of Pharmacy”, 21st ed., A. R. Gennaro, et al. Eds., c. 2005 by Lippincott Williams & Wilkins, (especially Part 7). As shown in Table 1, the typical dose ranges from 5 to 60 μg per adult. A more typical dose is from 20 to 40 μg per adult.

The following examples are presented by way of illustration and not limitation:

### Animal Model Examples

### Example 1

### Visceral Pain

In this model, the effects of morphine and TTX were compared. Morphine was administered at 1.25, 5, or 10 mg/kg and tetrodotoxin at 0, 3, or 4 μg/kg. The chemicals were administered subcutaneously to groups of 10 male mice. Thirty minutes later, 50 μL of a capsaicin solution (0.3% in water solution) was delivered to the colon via the rectum. The capsaicin-induced nociceptive behaviours exhibited by the mice were counted for a 30-minute period. The frequency of nociceptive behaviors was compared to that of a control group receiving only the capsaicin solution.

The effects were as follows:

Morphine (1.25, 5 and 10 mg/kg, s.c.): 54.8*, 92.5* and 100*%

Tetrodotoxin (3 and 4 μg/kg, s.c.): 30 and 58.8*%

(*: p<0.05, Anova followed Dunnett's test versus vehicle)

The results of the study suggest that a dose of 4 μg/kg of tetrodotoxin was as effective as 1.25 mg/kg of morphine.

### Example 2

### Postoperative Pain

### Thermal Stimulus

Tetrodotoxin was active in postoperative pain in rats, after acute or 4 days of pre-treatment.

### Acute Treatment

For this model, an incision was made in the hind paw of the rats. One hour later, tetrodotoxin was administered subcutaneously (0, 1, 4, or 8 μg/kg) to groups of 12 male rats. Thirty minutes later, a thermal stimulus was applied to the incised paw and the latency to withdraw the affected hind paw was recorded. The paw withdrawal latencies for each treatment group were measured. The hyperalgesia for each animal was calculated as the percentage of decrease of the latency time of withdrawing the incised versus the healthy hind paw of the same animal. The antinociceptive activity for each animal was calculated comparing its hyperalgesia value with the mean hyperalgesia of the control group (treated with vehicle).

The ED₅₀ of i.v. morphine in this model was 0.59 mg/kg.

The antinociceptive activity of tetrodotoxin was 8%, 20% and 35% following s.c. doses of 1, 4 and 8 μg/kg, respectively.

### Four Days of Pre-Treatment

The studied compounds (dypirone at 40 mg/kg and tetrodotoxin at 0, 3, 4 μg/kg) were administered subcutaneously to groups of 12 male rats twice daily for 4 days. On the morning of Day 5, an incision was made in the hind paw of the rats. One hour later, the compounds were administered again and 30 minutes later, a thermal stimulus was applied in the incised paw and the latency to withdraw the hind paws was recorded.

The antinociceptive activity was:

Dypirone (40 mg/kg, s.c., b.i.d.): 40%

Tetrodotoxin (3 and 4 μg/kg, s.c., b.i.d.): 35% and 39%, respectively.

### Example 3

### Inflammatory Pain

Experiments in this model takes the following steps:

a) Quantification of the baseline paw volume (plethysmometry) and baseline nociceptive threshold by the Randall-Selitto procedure (paw pressure) of male SD rats;
b) Drug treatment: TTX (2.5 μg/kg, s.c.) or vehicle;
c) After 1 h: Injection of 1% lambda carrageenan (0.1 mL) into the surface of the right hind paw;
d) Redetermination of paw volume and nociceptive threshold 3 h post injection of carrageenan.

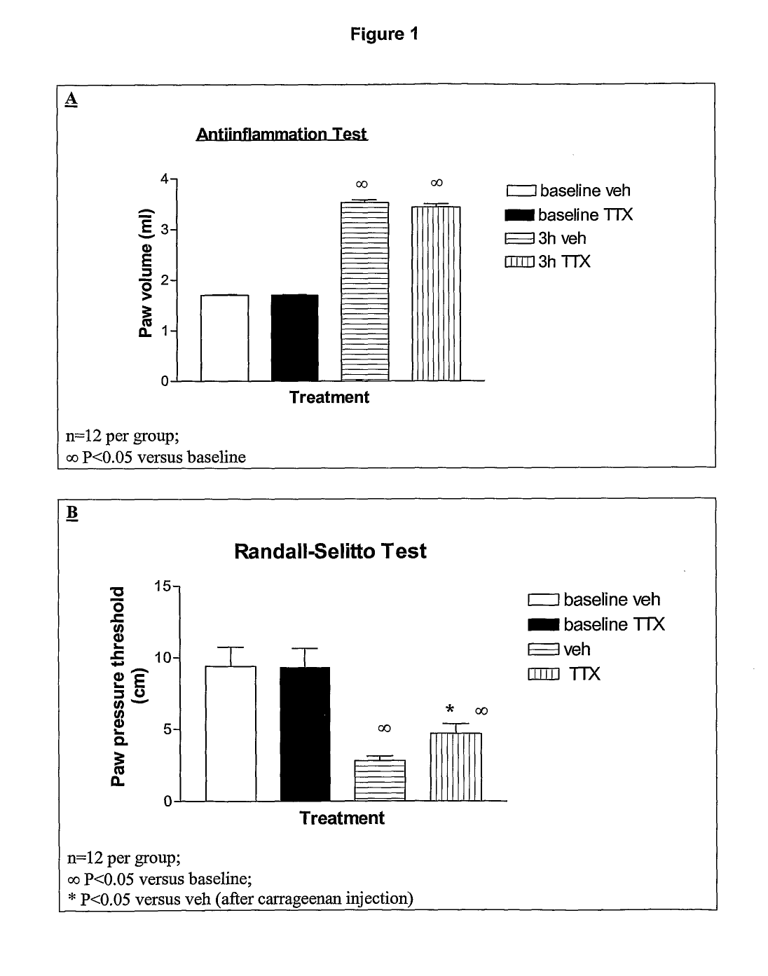

The result of the paw volume measurement, shown in FIG. 1-A, confirms the inflammatory response to carrageenan. The result of the Randall-Selitto test (paw pressure), shown in FIG. 1-B, shows that the TTX treated rats have a higher nociceptive threshold post carrageenan injection relative to the vehicle treated rats post carrageenan injection.

### Clinical Examples

Patients entered a four to seven day baseline period, following which subjects were admitted to hospital and admitted to a care facility to receive the drug on each of four consecutive days. TTX was formulated at a concentration of 30 μg/2 mL, and was administered by intramuscular injection. For each subject the study lasted up to six weeks from the start of screening. Patients who had experienced an analgesic effect were followed for a maximum of eight weeks or until the analgesic effect became inadequate. During the study subjects kept a daily pain diary (Brief Pain Inventory and/or Neuropathic Pain Scale). The Visual Analogue Scale and Edmonton Symptom Assessment Scale were also applied to assessing pain symptoms. Patients were classed as responders where they showed a decrease of 33% or more (BPI=3) in their worst 24 hour pain intensity, compared to baseline, for at least two consecutive days. Patients were classified as dramatic responders if they experienced a decrease in pain intensity of at least 50% on all global pain measures that extended well beyond the four day treatment period.

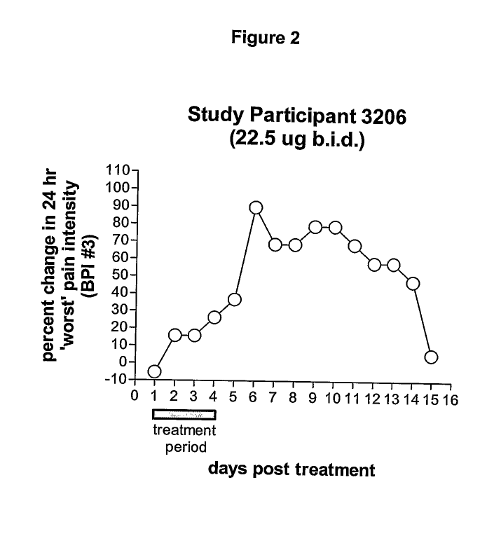

Case #1 (3206): A 51-year-old man developed visceral post radiotherapy treatment pain. The injury due to treatment resulted in tissue damage such that non-neuropathic pain was generated as a result of certain activities. He had flares of pain that were 10/10 in severity several times a day at baseline either spontaneous or triggered by raising his hands above his head as well as spells of spasms each day prior to tetrodotoxin. The patient received three 4-day treatment cycles of tetrodotoxin at a regime of 7.5 μg bid (first cycle), 22.5 μg bid (second cycle) and 30 μg tid (third cycle). Pain relief was achieved from days 3 to 9 inclusively in the first cycle. The patient had no flares of pain at all for several days but reported an increase of his underlying pain. On days 11-15 the patient was having spasm and flares of pain again, but they were not as severe as in the past. His underlying pain spasms increased to the point of pre-treatment from day 14, but were not worse than before the trial. The patient reported significant improvement in quality of life. He was able to engage in exercise and sleep again on his right side for the first time in nine years. Following that, the patient re-enrolled into a higher dose level: 22.5 μg b.i.d. (2nd cycle). After 4 days of treatment, he experienced a dramatic analgesic response from days 5 to 14. The most pain relief percentage reached 89% (See FIG. 2). Meanwhile, his Morphine immediate-release was discontinued from day 3 to day 12. Again this patient reported significant improvement in quality of life. He was able to move more comfortably, and decreased his medication for pain (BTA). In his 3rd treatment cycle, he experienced good respond from days 5 to 9 (40%˜50% pain relief percentage).

Case #2: A 36-year-old woman has chronic pancreatitis (idiopathic pancreatitis from 8 years of age). Her daily constant abdominal pain score was 5-6/10, increasing to 8/10 with exacerbation. She tried different pain medications, including anticonvulsant, antidepressant and local anesthetics. She received a TTX treatment cycle at the regime of 30 μg bid for 4 days. Her baseline worst pain score was 8/10. After one day of treatment, her pain score decreased to 7/10 and continued to go down to 4/10 at day 5. Her worst daily pain fluctuated between 3/10 and 6/10 during day 5 to day 19. Ten days after stopping TTX treatment, she still felt much improved, with less pain and more energy. She stated that she had not felt this well over one year.

Case #3: This Caucasian 70 year male had a history of malignant mesothelioma. The pathophysiology of his pain was neuropathic and visceral in origin. He reported severe pain in the left chest wall and back (left lower costal margin). He had constant burning and sharp pain, and flares of jabbing pain, which was inadequately managed by MS Contin, Dilaudid, and Celebrex. Prior to treatment with TTX, he reported an average of 6.7 out of 10 for his 24-hour ‘worst’ pain during the baseline period. Treatment with 30 μg of TTX, three times daily, for four days resulted in about 2-point decrease in his current and average pain intensity. Also during this period, he reported a reduced impact of pain on general activity, normal work, and sleep.

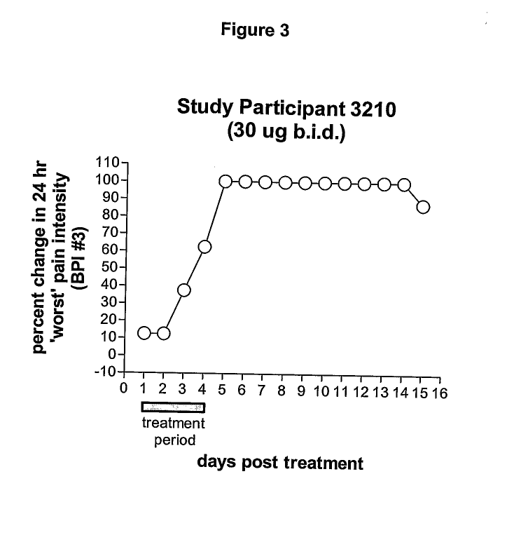

Case #4 (3210): This was a 68-year-old male with a history of prostate cancer. He had severe radiation-induced neuropathic pain of the perineum. His pain symptoms included allodynia and hyperpathia of the scrotum with a deep constant aching in the perineum. At baseline, this patient reported pain that was, on average, 8 out of 10 (24-hour ‘worst’ pain) despite taking the following medications: Oxycontin 20 mg p.o. TED, Oxycocet 5-10 mg q4-6 h prn, and Gabapentin 400 mg p.o. T. Following treatment with 30 μg bid TTX, this patient report complete relief of his pain by Day 5 which persisted until at least Day 15 (see FIG. 3). This patient also reported a complete reduction in the impact of pain on all aspects of his life beginning on Day 5 of treatment.

Case #5: This Caucasian 54-year old woman had a history of rectal cancer for which she received radiation therapy and resection of her colon. The radiation therapy produced a severe neuropathic pain syndrome that was characterized by a constant dull ache in the lumbar and pelvic regions, and leg, with sharp and jolting flares of her pain. She was initially enrolled into the 7.5 μg TTX dosage group, and experienced no analgesic response. Subsequently, she was enrolled into the 15 μg TTX twice daily group and then the 30 μg TTX twice daily group. The last two dosing regimens that she received produced an analgesic response. Her baseline ‘worst’ pain was consistently 10 out of 10. On the second day of treatment with the 15 μg dose her pain was reduced to a 7 out of 10, and then to 3 out of 10 for Days 3 and 4. Beginning of Day 5, her pain began to increase and reached baseline levels by Day 10. This patient's ‘current’ pain appeared to be more responsive to treatment with the 30 μg dose than her ‘worst’ pain. During treatment with this dose, she experienced a 30 to 70% reduction in her current pain intensity during Days 1-6.

In another case, the data is taken from a multi-centre, open-label, continuation trial of the efficacy and safety of tetrodotoxin in patients with stable but inadequately controlled moderate to severe pain associated with cancer. All patients who participated in this study (tetrodotoxin and placebo treated), and who would like to continue with tetrodotoxin treatment and met the inclusions/exclusion criteria, were eligible to receive the first Treatment Cycle for this continuation study.

| | | | | Total | | Duration of | |
|---|---|---|---|---|---|---|---|
| ID, | | | | TTX | | Analgesic | Duration |
| age, | Primary | | No. of | (μg) | | Response | of Study |
| sex | pain sites | Diagnosis | Cycles | Received | Response | (days) | (days) |
| 053, | Thoracic | Small | N = 5 | 1170 | Cycle 1: Responder | 23 days | 150 |
| 39 yrs, | and | intestine | | | Cycle 2: Responder | 13 days | |
| Female | abdominal | cancer | | | Cycle 3: Responder | 11 days | |
| | liver | | | | Cycle 4: Responder | 4 days* | |
| | right side | | | | Cycle 5: Responder | 15 days | |

*Data obtained only for D1-D5. Patient did not record pain score after day 5.

Case #6 (053): This 39-year-old Hispanic female had an intestinal cancer with metastasis to lung, abdomen, bone and liver. Her thoracic left shoulder pain was from bone metastasis (somatic) and abdominal pain on the right side from liver metastasis (visceral). She had received Methadone and Decadron in an attempt to manage her pain. But these failed. The pain intensity score of her abdominal liver pain was 10 when she received first cycle of TTX treatment (30 μg, twice daily for four days). After 4 days treatment, on day 5 her pain intensity was very much improved (impression of change=1) and the good response lasted 23 days. This patient repeated use of TTX for five cycles. Her study duration was 150 days and total response day was 66 days.

### Examples of Pharmaceutical Compositions

### Formulation Example 1

### Injectable Formulation

A formulated pharmaceutical composition of tetrodotoxin for injection, which injection may typically (by way of example and not of limitation) be intramuscular, intravenous, or subcutaneous, is shown in Table 2.

TABLE 2
Tetrodotoxin Formulation

| | |
|---|---|
| Tetrodotoxin | 15 mg |
| 0.5% dilute acetic acid | 1 mL |
| Acetic acid - acetate buffer | 50 mL (5% of the total volume of the |
| solution (pH = 3.5) | prepared pharmaceutical solution) |
| Water for injection. add to | 1000 mL |

The calculation of the formulation dosage of TTX for injection is based upon the results of pre-clinical pharmacology and pharmacodynamics studies. The calculation of the clinical pharmaceutical dosage is based upon the dosage effective in animals. In general, it is calculated as ⅕ of the effective animal dosage. 50, 60, and 70 kg are used as human body weights, respectively.

The TTX analgesic ID₅₀ (half inhibition dosage) in the acetic acid-induced twisting test in mice is 2.80 μg/kg (intramuscularly, IM). Accordingly, the recommended clinical dosage for humans is:

2.80μg/kg×(⅕)×50(60,70)kg=28.0(33.6,39.2)μg

The TTX effective dosage in the formalin-induced inflammation test in rats is 2.5 μg/mg (IM) (P<0.01). Accordingly, the recommended clinical dosage for humans is:

2.50μg/kg×(⅕)×50(60,70)kg=25.0(30.0,35.0)μg

It is also possible to calculate the initial clinical dosage based upon LD₅₀ value. Considering the results of pharmacodynamics studies, the clinical dosage can be calculated as 1/50 of the LD₅₀. 50, 60, and 70 kg are used as human body weights, respectively.

Based upon the results of pharmacology studies and related references, the dosage of TTX for injection used in the clinical study of the example in Table 2 is 30 μg in 2 mL.

### Orally Administerable Formulations

### Capsule Formulations

### Formulation Example 2

### Capsule

| | | |
|---|---|---|
| Tetrodotoxin (TTX) (powdered material) | 0.03 | mg |
| Colloidal silicon dioxide | 0.5 | mg |
| Magnesium stearate | 1.0 | mg |
| Lactose | 98.47 | mg |
| Total | 100 | mg |

### Formulation Example 3

### Capsule

| | | |
|---|---|---|
| Tetrodotoxin | 0.03 | mg |
| Colloidal silicon dioxide | 0.8 | mg |
| Magnesium stearate | 2.4 | mg |
| Lactose | 476.77 | mg |
| Total | 480 | mg |

### Tablet Formulations

### Formulation Example 4

### Tablet

| | | |
|---|---|---|
| Tetrodotoxin (TTX) (powdered material) | 0.03 | mg |
| Colloidal silicon dioxide | 0.5 | mg |
| Magnesium stearate | 1.0 | mg |
| Soidum croscarmelose | 5.0 | mg |
| Lactose | 93.47 | mg |
| Total | 100 | mg |

### Formulation Example 5

### Tablet

| | | |
|---|---|---|
| Tetrodotoxin (TTX) (powdered material) | 0.03 | mg |
| Sodium croscarmelose (AC-DI-SOL) | 40 | mg |
| Colloidal silica dioxide (AEROSYL 200) | 8 | mg |
| Magnesium stearate, NF | 16 | mg |
| POVIDONE K-30 | 40 | mg |
| Microcrystalline cellulose (AVICEL PH-102) | 346 | mg |
| Lactose monohydrate (FARMATOSE 200M) | 349.97 | mg |
| Total | 800 | mg |

### Formulation Example 6

### Tablet

| | | |
|---|---|---|
| Tetrodotoxin (TTX) (powdered material) | 0.03 | mg |
| Sodium croscarmelose (AC-DI-SOL) | 35 | mg |
| Colloidal silica dioxide (AEROSYL 200) | 3 | mg |
| Sodium stearate | 12 | mg |
| Polyethylene glycol 8000 | 30 | mg |
| Microcrystalline cellulose (Avicel PH-102) | 75 | mg |
| Lactose monohydrate (FARMATOSE 200M) | 420.97 | mg |
| OPADRY II ® | 24 | mg |
| Total | 600 | mg |

### Formulation Example 7

### Tablet (Humid Granulation)

| | | |
|---|---|---|
| Tetrodotoxin (TTX) (powdered material) | 0.03 | mg |
| Colloidal silicon dioxide | 0.5 | mg |
| Magnesium stearate | 1.0 | mg |
| POVIDONE K-30 | 5.0 | mg |
| Sodium carboxymethylstarch | 5.0 | mg |
| Microcrystalline cellulose | 20 | mg |
| Lactose | 68.47 | mg |
| Total | 100 | mg |

### Outwardly Solid Formulations

### Formulation Example 8

### Encapsulated Outwardly Solid Formulation

| | |
|---|---|
| Tetrodotoxin | 60 mg |
| 0.5% dilute acetic acid | 1 mL |
| Acetic acid - acetate buffer | 50 mL (5% of the total volume of the |
| solution (pH = 3.5) | prepared pharmaceutical solution) |
| Water for injection. add to | 1000 mL |

0.5 mL of this prepared solution were encapsulated in suitable consumable capsules and stored.

### Formulation Example 9

### A Tablet Ready to be Processed into an Enteric-Coated Formulation

| | | |
|---|---|---|
| Tetrodotoxin | 0.03 | mg |
| Dibasic Calcium Phosphate USP | 47.27 | mg |
| Avicel PH 101 | 50.0 | mg |
| NATROSOL 250 HHX | 1.0 | mg |
| CAB-O-SIL M5 | 0.5 | mg |
| Magnesium Stearate NF | 1.0 | mg |
| Yellow Lake F D & C No 6 | 0.2 | mg |
| Purified Water USP | | |
| (evaporates during the process) | | |
| Total | 100 | mg |

### Formulation Example 10

### An Enteric-Coated Version of Formulation Example 9

| | |
|---|---|
| Tablet according to Example 9 | 100 mg |
| Acryl-Eze yellow coating suspension House Std | 40.0 mg |

### Formulation Example 11

### A Tablet Ready to be Processed into a Coated Controlled-Release Formulation

| | | |
|---|---|---|
| Tetrodotoxin | 0.03 | mg |
| Dibasic Calcium Phosphate USP | 40.0 | mg |
| Avicel PH 101 | 47.27 | mg |
| NATROSOL 250 HHX | 10.0 | mg |
| CAB-O-SIL M5 | 0.5 | mg |
| Magnesium Stearate NF | 2.0 | mg |
| Blue F D & C No1 | 0.2 | mg |
| Purified Water USP | | |
| (evaporates during the process) | | |
| Total | 100 | mg |

### Formulation Example 12

### A Coated Controlled-Release Version of Formulation Example 11

| | |
|---|---|
| Tablet according to Example 11 | 100 mg |
| SURETERIC Blue suspension House Std | 20 mg |
| 90/10 SURELEASE/OPADRY clear suspension | 30 mg |

### Formulation Example 13

### A Tablet Ready to be Processed into a Coated Formulation

| | | |
|---|---|---|
| Tetrodotoxin | 0.03 | mg |
| Dibasic Calcium Phosphate USP | 46.47 | mg |
| Avicel PH 101 | 50 | mg |
| AC-DI-SOL | 2.0 | mg |
| CAB-O-SIL M5 | 0.5 | mg |
| Magnesium Stearate NF | 1.0 | mg |
| Purified Water USP | | |
| (evaporates during the process) | | |
| Total | 100 | mg |

### Formulation Example 14

### A Coated Version of Formulation Example 13

| | |
|---|---|
| Tablet according to Example 13 | 100 mg |
| OPADRY II coating suspension House Std | 20 mg |

With the guidance provided herein, once the required parameters of a composition or method are known, those skilled in the art will be readily able to determine the amounts and proportions of active components and other components required to manufacture a required dosage form, manufacture a kit or composition, or use the methods and compositions disclosed. The foregoing embodiments have been described in detail by way of illustration and example for purposes of clarity and understanding. As is readily apparent to one skilled in the art, the foregoing are only some of the methods and compositions that illustrate the possible embodiments. It will be apparent to those of ordinary skill in the art that a range of equivalents, variations, changes, modifications and alterations may be applied to the compositions and methods described herein without departing from the true spirit, concept and scope of the invention.

1. A method for the treatment of visceral pain in a mammal, the method comprising administering to a mammal in need thereof an effective amount of a sodium channel blocker
5 that binds to the SS 1 or SS2 site of the extracellular region of an alpha subunit of a sodium channel.

2. The method according to claim 1 wherein the visceral pain is associated with chronic pancreatitis.

3. The method according to claim 1 wherein the visceral pain is perineal pain.

4. The method according to claim 1 wherein the visceral pain is pelvic pain.

5. The method according to claim 1 wherein the visceral pain is scrotal pain.

6. The method according to claim 1 wherein the visceral pain is chest pain.

7. The method according to claim 6 wherein the chest pain is pain of the chest wall.

8. The method according to claim 1, wherein the visceral pain is associated with irritable bowel syndrome.

9. The method according to claim 1, wherein the visceral pain is associated with gastrointestinal dyspepsia.

10. The method according to claim 1, wherein the visceral pain is associated with interstitial cystitis.

11. The method according to claim 1 wherein the visceral pain is associated with gall bladder dysfunction.

12. The method according to claim 1, wherein the visceral pain is associated with vulvodynia.

13. The method according to claim 1, wherein the visceral pain is associated with urethral syndrome.

14. The method according to claim 1, wherein the visceral pain is associated with endometriosis.

15. The method according to claim 1, wherein the visceral pain is associated with dysmenorrhea.

16. The method according to claim 1, wherein the visceral pain is associated with prostatodynia.

17. The method according to claim 1, wherein the visceral pain is penile pain.

18. The method according to claim 1 wherein the pain is caused by therapy.

19. The method according to claim 18 wherein the therapy comprises operative therapy.

20. The method according to claim 18 wherein the therapy comprises radiation therapy.

21. The method according to claim 18 wherein the therapy comprises chemotherapy.

22. The method according to claim 1 wherein the visceral pain is inflammatory pain.

23. The method according to claim 1 wherein the visceral pain is acute pain.

24. The method according to claim 1 wherein the visceral pain is chronic pain.

25. The method according to claim 1 further comprising formulating a medicament comprising the sodium channel blocker.

26. The method according to claim 1 wherein the sodium channel blocker is selected from the group consisting of: tetrodotoxin, saxitoxin, and derivatives or analogues of tetrodotoxin and saxitoxin.

27. The method according to claim 26 wherein the sodium channel blocker is tetrodotoxin or an analogue or derivative thereof.

28. The method according to claim 26 wherein the sodium channel blocker is selected from the group consisting of tetrodotoxin, anhydro-tetrodotoxin, tetrodaminotoxin, methoxytetrodotoxin, ethoxytetrodotoxin, deoxytetrodotoxin, epi-tetrodotoxin and tetrodonic acid.

29. The method according to claim 26 wherein the sodium channel blocker is tetrodotoxin.

30. The method according to claim 26, wherein the sodium channel blocker is isolated from a fish.

31. The method according to claim 30 wherein the fish is a puffer fish.

32. The method according to claim 1 wherein sodium channel blocker is produced by synthesis or fermentation.

33. The method according to claim 1 comprising using a kit to administer said sodium channel blocker, said kit comprising said sodium channel blocker and instructions to use it to treat pain.

34. The method according to claim 1 further wherein the sodium channel blocker is administered orally.

35. The method according to claim 34 wherein said oral administration is sublingual, buccal or transmucosal administration.

36. The method according to claim 1 wherein the sodium channel blocker is administered by injection.

37. The method according to claim 33 wherein said sodium channel blocker is administered in an amount of between about 5 μg and about 300 μg per unit dose.

38. The method according to claim 37 wherein said amount is between about 5 μg and about 50 μg.

39. The method according to claim 34 wherein said sodium channel blocker is administered over a period of between about one and about five days.

40. The method according to claim 34, wherein said sodium channel blocker is administered in multiple treatment cycles.
